(19) United States
(12) Plant Patent Application Publication
(10) Pub. No.: US 2016/0295760 P1
(43) Pub. Date: Oct. 6, 2016

(54) Pear 'HW624'

(50) ;

(71) Applicant: Her Majesty the Queen in Right of Canada, as represented by the Minister of Agriculture & Agri-, Ottawa (CA)

(72) Inventor: David M. Hunter, St. Catharines (CA)

(21) Appl. No.: 14/545,134

(22) Filed: Mar. 31, 2015

(51) Int. Cl. A01H 5/00

(52) U.S. Cl. PLT/176

(57) ABSTRACT

The invention is a new and distinct pear tree variety denominated ‘HW624’. The new pear tree is characterized by producing heavy crops of medium-to large-sized fruit, picked about 2 weeks after ‘Bartlett’. Fruits are green when harvested, turning yellow as fruits ripen, and there is a medium red blush where exposed to the sun. There were no natural fire blight infections on the seedling tree, and results from controlled inoculations indicate a high level of resistance to fire blight. The tree is also highly resistant to pear psylla.

### LATIN NAME OF THE GENUS AND SPECIES OF THE PLANT CLAIMED

Pyrus communis

### VARIETY DENOMINATION

‘HW624’

### BACKGROUND OF THE INVENTION

The new and distinct pear tree described and claimed herein originated from a controlled cross between ‘Harrow Sweet’ and NY10353 made in 1988 at Agriculture and Agri-Food Canada Harrow Research Station in Harrow, Ontario. Pedigree of ‘HW624’ is shown in FIG. 1.

‘HW624’ was selected as a hybrid seedling (H8806-1) in 1995 and propagated by budding on pear seedling rootstocks. Trees were planted in an evaluation orchard at the Agriculture and Agri-Food Canada Research Farm at Jordan Station in 1999. This selection was advanced and tested in regional trials in cooperation with the Western Ontario Fruit Testing Association beginning in 2000.

### SUMMARY OF THE INVENTION

On the new pear tree ‘HW624’, the profile of the sides of the fruit of ‘HW624’ is straight whereas they are concave on the reference varieties Bosc (R1), Harrow Sweet (R2), AC Harrow Crisp (R3) and Bartlett (R4). The ground colour of the skin of ‘HW624’ is yellow whereas it is yellow green on ‘Bosc’, ‘Harrow Street’ and ‘Bartlett’. ‘HW624’ has a medium to large area of over colour of the skin whereas there is none on ‘Bosc’, small to medium area on ‘Harrow Street’ and very small on ‘Bartlett’. The over colour of ‘HW624’ is dark red whereas it is orange red on ‘Harrow Street’, ‘AC Harrow Crisp’ and ‘Bartlett’. ‘HW624’ has no russetting around the eye basin, on the cheeks or around the stalk attachment of the fruit whereas ‘Bosc’ has a large to very large area. The fruit stalk of ‘HW624’ is thin whereas it is medium thickness on ‘Harrow Street’ and ‘AC Harrow Crisp’ and thick on ‘Bartlett’. ‘HW624’ has weak curvature of the fruit stalk whereas the curvature is medium to strong on ‘Bosc’, medium on ‘AC Harrow Crisp’ and absent on ‘Bartlett’. The attitude of the sepals on the fruit of ‘HW624’ is preading whereas it is erect on ‘Bosc’ and ‘Harrow Street’ and converging on ‘AC Harrow Crisp’ and ‘Bartlett’. ‘HW624’ is resistant to moderately resistant to fireblight (Erwinia amylovora) whereas ‘Bosc’ is moderately susceptible and ‘Bartlett’ is susceptible. ‘HW624’ is resistant to moderately resistant to pear psylla (Cacopsylla pyricola Foerst) whereas the reference varieties are susceptible.

The following are major distinguishing characteristics. ‘HW624’ is a mid-late season pear cultivar for the fresh market. ‘HW624’ produces heavy crops of medium-to large-sized fruit, picked about 2 weeks after ‘Bartlett’. Fruits are green when harvested, turning yellow as fruits ripen, and there is a medium red blush where exposed to the sun. The tree has consistently produced heavy crops, with no evidence of biennial bearing. There were no natural fire blight infections on the seedling tree, and results from controlled inoculations indicate a high level of resistance to fire blight. The tree is also highly resistant to pear psylla (Cacopsylla pyricola Foerst). In years when orchard populations of psylla have been high, trees of ‘HW624’ have had no noticeable presence of this insect pest. ‘HW624’ is not graft-compatible with quince rootstocks. The characteristics most useful in distinguishing ‘HW624’ are fruit characteristics and resistance to fire blight and resistance to pear psylla.

### BRIEF DESCRIPTION OF THE DRAWING

The accompanying photographic illustration illustrates in full color ‘HW624’. The colors are as nearly true as reasonably possible in color representation of this type. Colors in the photographs may differ from the color values cited in the detailed botanical description below, which accurately describes the colors of the new pear tree.

FIG. 1 shows pedigree of ‘HW624’.

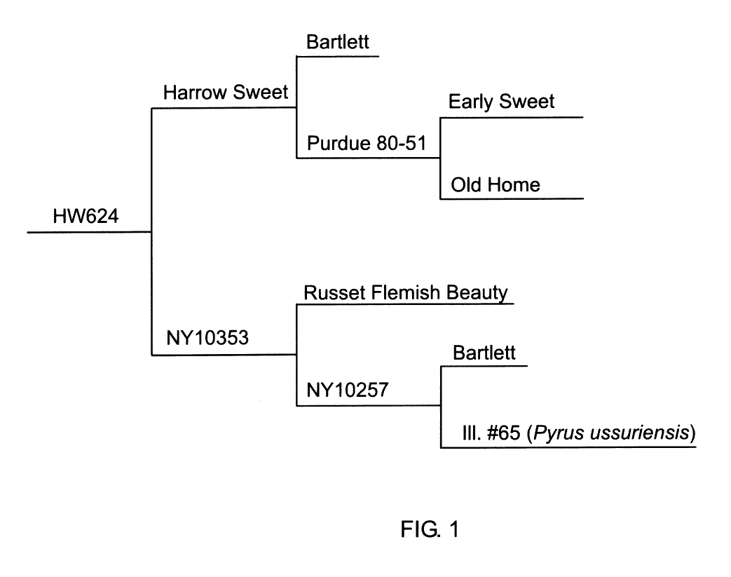

FIG. 2 shows five examples of the fruit of ‘HW624’ compared to the fruit of Bosc (R1), Harrow Sweet (R2), AC Harrow Crisp (R3) and Bartlett (R4).

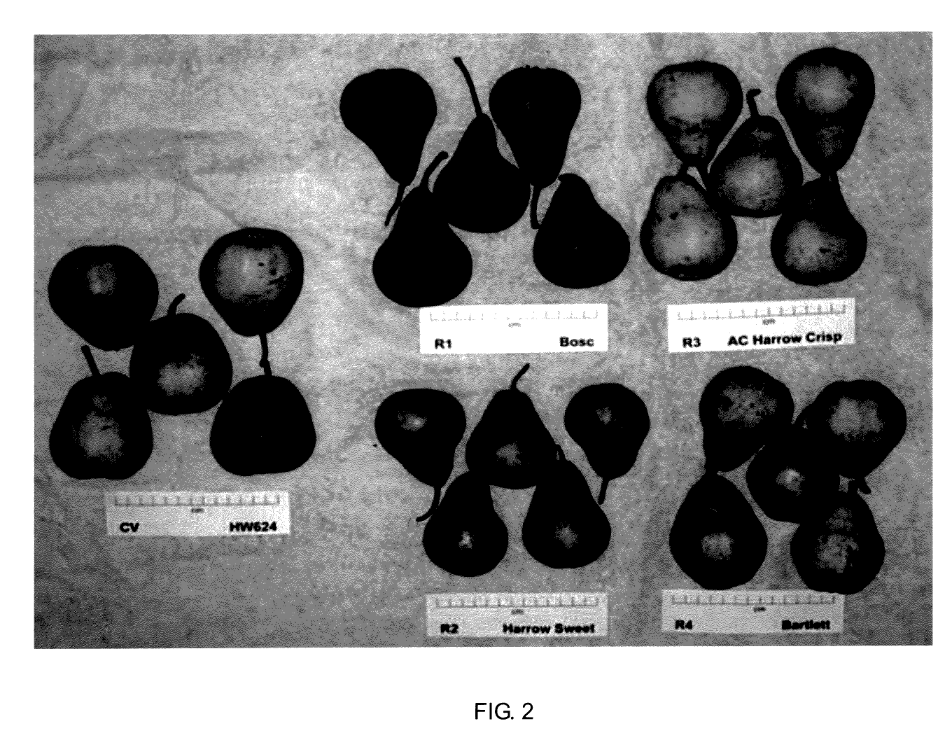

### DETAILED BOTANICAL DESCRIPTION OF THE INVENTION

The ‘HW624’ variety is uniform and stable. Asexual propagation maintains uniformity and stability. No off-types, variants or mutations have been found to date. Virus-tested ‘HW624’ is maintained at the Canadian Food Inspection Agency, Centre for Plant Health in North Saanich, British Columbia.

The R.H.S. Colour Chart of The Royal Horticultural Society has been used for colour identification of fruit and shoot. The description is based on observation of four trees grafted on seedling rootstock originally established during the 1999 growing season. Comparisons were made with Bosc (R1), Harrow Sweet (R2), AC Harrow Crisp (R3) and Bartlett (R4). Four trees of each variety grafted on Bartlett open pollinated seedling rootstock were spaced about 3 metres apart with about 4.5 metres between rows and were observed during the 2009-2010 growing seasons at the Jordan Road Farm of Agriculture and Agri-Food Canada Research Centre in Jordan Station, Ontario. Fire blight evaluations and observations were conducted in 1988-2000 at the Agriculture and Agri-Food Canada Harrow Research Centre.

| | | | | |
|---|---|---|---|---|
| ‘HW624’ | R1 | R2 | R3 | R4 |

Tree:

| | | | | | |
|---|---|---|---|---|---|
| Vigor | medium | very | medium | strong | medium |
| | to strong | strong | to strong | | |
| Branching | strong | medium | strong | medium | medium |
| | | to strong | | to strong | |
| Habit | spreading | spreading | spreading | spreading | spreading |
| | to | | | | |
| | drooping | | | | |

Shoot-One-year-old:

| | | | | | |
|---|---|---|---|---|---|
| Growth | wavy | wavy | wavy | straight | straight |
| | | | | to | to |
| Length of | | | | wavy | wavy |
| internode | | | | | |
| Mean | 3.42 | 4.28 | 3.02 | 3.26 | 3.55 |
| Std. deviation | 0.37 | 0.67 | 0.48 | 0.58 | 0.46 |
| Predominant | brown | brown | orange | medium | orange |
| colour on | purple | purple | brown | brown | brown |
| sunny side | | | | | |
| Number of | many | many | medium | medium | medium |
| lenticels | | | to many | to many | to many |
| Shape of apex | acute | acute | acute | acute | acute |
| of vegetative | | | | | |
| bud | | | | | |
| Position of | slightly | slightly | slightly | slightly | adpressed |
| vegetative | held out | held out | held out | held out | |
| bud in relation | | to | | | |
| to shoot | | markedly | | | |
| | | held out | | | |
| Size of bud | medium | small to | small to | medium | small to |
| support | | medium | medium | | medium |

Shoot-Young:

| | | | | | |
|---|---|---|---|---|---|
| Anthocyanin | very | strong to | medium | medium | medium |
| colouration of | strong | very | | to strong | |
| growing tip | | strong | | | |
| (during rapid | | | | | |
| growth) | | | | | |
| Intensity of | weak to | weak to | weak to | weak to | medium |
| pubescence | medium | medium | medium | medium | |
| (upper third) | | | | | |

Leaf-Blade:

| | | | | | |
|---|---|---|---|---|---|
| Attitude in | outwards | down- | outwards | outwards | outwards |
| relation to | | wards | | | |
| shoot | | | | | |
| Length (cm) | | | | | |
| Mean | 6.5 | 7.1 | 7.1 | 6.3 | 6.5 |
| Std. deviation | 1.0 | 1.3 | 1.2 | 0.8 | 0.7 |
| Width (cm) | | | | | |
| Mean | 4.3 | 4.3 | 3.8 | 3.8 | 3.9 |
| Std. deviation | 0.7 | 1.0 | 0.8 | 0.7 | 0.6 |
| Length/Width | small to | medium | medium | medium | medium |
| ratio | medium | | to large | | |
| Shape of base | right- | obtuse | right- | right- | right- |
| | angled | | angled | angled | angled |
| | | | | to | to |
| | | | | obtuse | obtuse |
| Shape of apex | obtuse | right- | right- | right- | right- |
| (excluding | | angled | angled | angled | angled |
| pointed tip) | | | | | to |
| | | | | | obtuse |
| Length of | short | short | short | short to | short |
| pointed tip | | | | very | |
| | | | | short | |
| Incisions of | bluntly | crenate | bluntly | bluntly | bluntly |
| margins | serrate | | serrate | serrate | serrate |
| (upper half) | | | | | |
| Depth of inci- | shallow | shallow | shallow | shallow | shallow |
| sions of | | | | | |
| margin | | | | | |
| Curvature of | very | weak to | weak | weak | very |
| longitudinal | weak | medium | | | weak |
| axis | | | | | |

Leaf-Petiole:

| | | | | | |
|---|---|---|---|---|---|
| Length (cm) | | | | | |
| Mean | 2.7 | 2.2 | 2.4 | 2.1 | 2.0 |
| Std. deviation | 0.6 | 1.0 | 0.7 | 0.7 | 0.8 |
| Presence of | present | Absent or | present | present | present |
| stipules | | weakly | | | |
| Distance of | medium | medium | short to | short to | short |
| stipules from | to long | | medium | medium | |
| basal attach- | | | | | |
| ment of | | | | | |
| petiole | | | | | |

Flower:

| | | | | | |
|---|---|---|---|---|---|
| Time of be- | early | medium | early to | medium | medium |
| ginning of | | to late | medium | to late | |
| flowering | | | | | |
| Location of | mainly | mainly | mainly | mainly | mainly |
| flower bud on | on spurs | on spurs | on spurs | on spurs | on spurs |
| shoot | | | | | |
| Length of - | | | | | |
| flower bud | | | | | |
| (cm) | | | | | |
| Mean | 5.4 | 5.9 | 5.5 | 6.3 | 4.1 |
| Std. deviation | 0.7 | 0.5 | 0.6 | 0.4 | 0.3 |
| Sepal length | | | | | |
| (cm) | | | | | |
| Mean | 5.9 | 8.3 | 6.7 | 7.9 | 7.0 |
| Std. deviation | 0.6 | 0.7 | 0.7 | 1.1 | 1.2 |
| Attitude of | recurved | recurved | recurved | recurved | spreading |
| sepals in rela- | | | | | to |
| tion to corolla | | | | | recurved |
| Arrangement | not | not | touching | touching | touching |
| of petals | touching | touching | to | | to |
| | to | | over- | | over- |
| | touching | | lapping | | lapping |
| Position of | same | same | above | same | same |
| stigma in | level | level | | level | level |
| relation to | | | | | |
| stamens | | | | | |
| Size of petal | small to | medium | medium | small to | medium |
| | medium | | | medium | |
| Shape of | circular | ovate | circular | circular | circular |
| petal (exclud- | to broad | | | | |
| ing the claw) | ovate | | | | |
| Shape of base | cuneate | cuneate | rounded | rounded | Rounded |
| of petal (ex- | to | | | | |
| cluding the | rounded | | | | |
| claw) | | | | | |
| Length of | short | medium | medium | very short | short |
| claw of petal | | | | | |

Fruit:

| | | | | | |
|---|---|---|---|---|---|
| Length (cm) | | | | | |
| Mean | 8.7 | 10.2 | 9.1 | 9.1 | 9.0 |
| Std. deviation | 1.0 | 0.9 | 0.9 | 0.8 | 0.9 |
| Maximum | | | | | |
| diameter (cm) | | | | | |
| Mean | 6.9 | 7.0 | 6.6 | 6.8 | 6.8 |
| Std. deviation | 0.6 | 0.5 | 0.6 | 0.6 | 0.6 |
| Length/Diam- | small to | medium | medium | medium | small to |
| eter ratio | medium | to large | | | medium |
| Position of | slightly | slightly | slightly | slightly | in |
| maximum | towards | towards | towards | towards | middle |
| diameter | calyx | calyx to | calyx | calyx | |
| | | clearly | | | |
| | | towards | | | |
| | | calyx | | | |
| Size | large | large | medium | large | medium |
| | | | | | to large |
| Symmetry (in | slightly | slightly | slightly | slightly | sym- |
| longitudinal | asym- | asym- | asym- | asym- | metric |
| section) | metric | metric to | metric | metric | to |
| | | strongly | | | slightly |
| | | asym- | | | asym- |
| | | metric | | | metric |
| Profile of sides | straight | concave | concave | concave | concave |
| Ground colour | yellow | yellow | yellow | yellow | yellow |
| of skin | | green | green | | green |
| Relative area | medium | absent | small to | medium | between |
| of over colour | to large | or very | medium | | very |
| | | small | | | small |
| | | | | | and |
| | | | | | small |
| Hue of over | dark red | | orange | orange | orange |
| colour | | | red | red | red |
| Relative area | absent | large to | absent | absent | absent |
| of russet | or very | very | or very | or very | or very |
| around eye | small | large | small | small | small |
| basin | | | | | |
| Relative area | absent | large to | absent | absent | absent |
| of russet on | or very | very | or very | or very | or very |
| cheeks | small | large | small | small | small |
| Relative area | absent | large to | absent | small | very |
| of russet | or very | very | or very | | small |
| around stalk | small | large | small | | to small |
| attachment | | | | | |
| Length of | | | | | |
| stalk (mm) | | | | | |
| Mean | 3.2 | 5.0 | 3.4 | 3.7 | 2.8 |
| Std. deviation | 0.7 | 0.7 | 0.6 | 0.9 | 0.6 |
| Thickness of | thin | thin to | medium | medium | thick |
| stalk | | medium | | | |
| Curvature of | weak | medium | weak to | medium | absent |
| stalk | | to strong | medium | | or very |
| | | | | | weak |
| Attitude of | oblique | oblique | oblique | straight | straight |
| stalk in rela- | | | | to | to |
| tion to axis | | | | oblique | oblique |
| of fruit | | | | | |
| Depth of stalk | medium | absent | shallow | medium | medium |
| cavity | to deep | or very | | | |
| | | shallow | | | |
| Attitude of | spreading | erect | erect | con- | con- |
| sepals (at | | | | verging | verging |
| harvest) | | | | | |
| Eye basin (at | present | present | present | present | present |
| harvest) | | | | | |
| Depth of eye | medium | shallow | shallow | shallow | deep |
| basin (at | to deep | | | to | |
| harvest) | | | | medium | |
| Width of eye | medium | narrow | medium | medium | narrow |
| basin (at | | to | to broad | | |
| harvest) | | medium | | | |
| Relief of area | slightly | slightly | slightly | smooth | em- |
| around eye | ribbed | ribbed | ribbed | | bossed |
| (at harvest) | | | | | |
| Texture of | very | very | very | very | very |
| flesh | fine | fine | very | very | very |
| | | | fine | fine | fine |
| Firmness | medium | medium | soft to | medium | medium |
| of flesh | to firm | | medium | | to firm |
| Juiciness | medium | juicy | juicy to | medium | juicy |
| of flesh | to juicy | | very | to juicy | |
| | | | juicy | | |
| Seed shape | ovate | ovate | elliptic | elliptic | ovate |
| Time of | late | very | very | medium | medium |
| maturity for | | late | late | | to late |
| consumption | | | | | |

Disease/Pest Reactions:

| | | | | | |
|---|---|---|---|---|---|
| Fire blight | resistant | moder- | resistant | resistant | suscep- |
| (Elwininia | to moder- | ately sus- | to moder- | to moder- | tible |
| amylovora) | ately | ceptible | ately | ately | |
| | resistant | | resistant | resistant | |
| Pear psylla | resistant | suscep- | suscep- | suscep- | suscep- |
| (Cacopsylla | to moder- | tible | tible | tible | tible |
| pyricola) | ately | | | | |
| | resistant | | | | |

The ‘HW624’ variety has not been observed under all possible environmental conditions to date. Accordingly, it is possible that the phenotypic expression may vary somewhat with changes in light intensity and duration, cultural practices, and other environmental conditions.

What is claimed:

1. A new and distinct variety of pear tree substantially as herein illustrated and described.
